SEQUENCE LISTING (12 sequences)

SEQ ID NO: 1 - DNA, 19 bp, synthetic construct
  source 1..19: mol_type = other DNA
tggctactaccgaagagct

SEQ ID NO: 2 - DNA, 20 bp, synthetic construct
  source 1..20: mol_type = other DNA
tgcagcattgttagcaggat

SEQ ID NO: 3 - DNA, 41 bp, synthetic construct
  source 1..41: mol_type = other DNA
tctggcccagttcctaggtagtccagacgaattcgtggtgg

SEQ ID NO: 4 - DNA, 40 bp, synthetic construct
  source 1..40: mol_type = other DNA
agacggcatcatatgggttgcacgggtgccaatgtgatct

SEQ ID NO: 5 - DNA, 19 bp, synthetic construct
  source 1..19: mol_type = other DNA
tttcggaagagacaggtac

SEQ ID NO: 6 - DNA, 22 bp, synthetic construct
  source 1..22: mol_type = other DNA
aggaactctagaagaattcaga

SEQ ID NO: 7 - DNA, 42 bp, synthetic construct
  source 1..42: mol_type = other DNA
cgcagtaaggatggctagtgtacgtacttctttttcttgctt

SEQ ID NO: 8 - DNA, 43 bp, synthetic construct
  source 1..43: mol_type = other DNA
tcgattgtgtgcgtactgctgtttttaacacgagagtaaacgt

SEQ ID NO: 9 - DNA, 16 bp, synthetic construct
  source 1..16: mol_type = other DNA
attaccccctgcatac

SEQ ID NO: 10 - DNA, 18 bp, synthetic construct
  source 1..18: mol_type = other DNA
aaccaagtaacattggaa

SEQ ID NO: 11 - DNA, 40 bp, synthetic construct
  source 1..40: mol_type = other DNA
ctgaaaactttgtcagggtaatactaattctttcacacgt

SEQ ID NO: 12 - DNA, 41 bp, synthetic construct
  source 1..41: mol_type = other DNA
atcctcagttttacattcaactagaaaggtaagaacaagtc

(19) United States
(12) Patent Application Publication
(10) Pub. No.: US 2025/0171864 A1
(43) Pub. Date: May 29, 2025

(54) RAPID METHOD FOR ROOM TEMPERATURE REVERSE TRANSCRIPTION LOOP-MEDIATED ISOTHERMAL AMPLIFICATION (RT-LAMP) AND REAGENT KIT

(71) Applicant: D-NOME PRIVATE LIMITED, Hyderabad (IN)

(72) Inventors: SUJOY DEB, Hyderabad (IN); DIVYA SRIRAM, Hyderabad (IN)

(73) Assignee: D-NOME, Hyderabad (IN)

(21) Appl. No.: 18/712,169

(22) PCT Filed: Nov. 23, 2022
(86) PCT No.: PCT/IB2022/061332

§ 371 Date: May 21, 2024

(30) Foreign Application Priority Data
Nov. 24, 2021 (IN) 202141054206

(51) Int. Cl. C12Q 1/70; C12N 15/10; C12Q 1/6806; C12Q 1/6844

(52) U.S. Cl. CPC C12Q 1/701; C12N 15/1096; C12Q 1/6806; C12Q 1/6844

(57) ABSTRACT

The present invention relates to a rapid method to perform reverse transcription loop-mediated isothermal amplification (RT-LAMP) and LAMP at room temperature between 25-42° C., more specifically at 25-37° C., to detect RNA/DNA in a sample and a reagent kit thereof. Further, the invention relates to an in vitro method to detect SARS-CoV2 using RT-LAMP at room temperature between 25-42° C., more specifically at 37° C. The reagent kit comprises of enzymes/proteins—Klenow exo−/−, ApaI, High fidelity Taq Pol, Rpa32, StpA, AMV-RT for reverse transcriptase; buffer composition of—Tris-HCl, MgSO₄, KCl, DTT, PEG, DMSO, 1 mM dNTPs each, at least 4 primers, and fluorescent or colorimetric dye.

### PRIORITY PARAGRAPH

The Application claims priority to PCT application PCT/IB2022/061332 filed on 23 Nov. 2022 titled “Rapid method for room temperature reverse transcription loop-mediated isothermal amplification (RT-LAMP) and reagent kit” that claims priority to the Indian provisional application 202141054206 Titled “Rapid method for room temperature reverse transcription loop-mediated isothermal amplification (RT-LAMP) and reagent kit” filed on 24 Nov. 2021. Both priority applications are fully incorporated herein by reference.

### REFERENCE TO AN ELECTRONIC SEQUENCE LISTING

The contents of the electronic sequence listing (Sequencelisting-D-Nome.xml; Size: 12 kb; and Date of Creation: May 21, 2024) is herein incorporated by reference in its entirety.

### FIELD OF THE INVENTION

The present invention relates to a rapid method to perform reverse transcription loop-mediated isothermal amplification (RT-LAMP) at room temperature to ambient temperature between 25-42° C., more specifically between 25-37° C., and a reagent kit thereof. Further, the invention relates to an in vitro method to detect SARS-CoV2 using RT-LAMP between 25-42° C., more specifically between 25-37° C. and a reagent kit thereof.

### BACKGROUND OF THE INVENTION

Disease diagnosis is one of the critical steps in disease management. Method or techniques to detect diseases (infectious diseases, genetic diseases, cancers, etc.) generally suffer from either specificity or sensitivity of detection. DNA amplification is a powerful platform technology for diagnostics applications due to its speed, sensitivity, specificity, cost-effectiveness and adaptability. The most widely used amplification method is the polymerase chain reaction (PCR). PCR requires cycling of temperature for several rounds, thermal cycling, for denaturation (90-98° C.), annealing (50-65° C.), and extension (65-80° C. based on the Polymerase activity), followed by final steps of extension, and hold (4-8° C.). Generally, Taq polymerase is used for DNA PCR which works best at 65-75° C.). This technique requires an expensive thermal cycler, expensive reagents, and skilled labor. The currently used technique which is a gold-standard technique in diagnoses is detection of nucleic acids using quantitative Real time Polymerase Chain Reaction (qRT-PCR) which has high specificity and sensitivity. But the major drawbacks of this technique include requirement of expensive equipment, reagents, and skill sets. This limits its use to scientific laboratories and becoming expensive for patients. Rapid tests on other hand that rely on detection of antibodies against pathogenic proteins, are good as point-of-care devices but not accurate, not sensitive enough, and hence unreliable.

Other common techniques used for detection of nucleic acids are the isothermal DNA amplification technologies, loop-mediated isothermal amplification (LAMP) and Recombinase Polymerase Amplification (RPA). LAMP and RPA are isothermal techniques conducted at a constant temperature. In LAMP, the DNA polymerases enzymatically separate the DNA strands instead of relying on elevated temperatures as in PCR. RPA utilizes enzymes such as DNA recombinase to facilitate primer annealing while single stranded DNA binding proteins hold the DNA stands apart to allow the DNA polymerase to initiate DNA synthesis. These techniques are advantageous over normal PCR as they obviate the requirement of thermal cycling. The device required for the reaction can be significantly smaller and cheaper as it will have a lower power demand than a thermal cycler. Second, isothermal amplification is more sensitive and faster than PCR as it does not rely on discrete thermal cycles like PCR, but rather involves continuous amplification.

LAMP uses four (or six) different primers which bind to six (or eight) different regions on the target gene making it highly specific. This primer set consists of two outer (F3 and B3) primers, two inner primers (forward inner primer (FIP) and backward inner primer (BIP)) and loop primers (loop forward and loop backward). LAMP reaction can be done simply at an isothermal condition by using Bst DNA polymerase which has high-displacement activity. Recently, LAMP was used for the detection of a wide range diseases caused by viruses, protozoans, and other disease causing parasites. Reverse transcription loop-mediated isothermal amplification (RT-LAMP) is a one-step nucleic acid amplification technique used by taking a step further and adding LAMP with a reverse transcriptase enzyme to allow RNA detection. RT-LAMP enables detecting RNA viruses such as HIV, coronavirus etc.

Prior art citations related to development of RT-LAMP for detecting infectious diseases including coronaviruses:

JP2012024039A discloses primer set for specifically amplifying a coronavirus-originated nucleic acid for LAMP amplification. The RT-LAMP is carried at isothermal reaction temperature of 62-65° C. using Bst DNA polymerase, and AMV Reverse transcriptase.

Similarly, many other citations provide method for detecting viral genomes using RT-LAMP at isothermal reaction temperature of 63° C. and inactivation temperature ranging from 80-95° C.
U.S. Ser. No. 11/072,822B2 discloses use of Tin(exo-) DNA polymerase and/or Bsm DNA polymerase;
CN111394520A discloses detecting new coronavirus based on RT-L AMP technology using Bst DNA polymerase, and Warmstart reverse transcriptase; and
CN111411173A discloses rapid detection RT-LAMP kit using Bst DNA polymerase, and AMV Reverse transcriptase.

However, LAMP technique suffers from high rates of false positives. Also, LAMP techniques are performed at 60-65° C. with an inactivation step at 80° C., thus requiring a heat block or PCR machine to perform the test.

The current need is development of an RT-LAMP technique for detecting infectious diseases including coronaviruses carried out at more feasible temperatures such as standard room temperatures, ambient temperatures or body temperatures between 25-42° C., at an average of 37° C., which makes it less expensive, sensitive, and which has high specificity. This requires a novel combination of reaction mixture composition that allows RT-LAMP at lower temperatures such as 25° C.-37° C. and elimination of heat inactivation step, thus removing heating requirements. Further, there is the need to reduce false positives by standardizing concentrations of buffer components and primers.

### OBJECT(S) OF THE INVENTION

Accordingly, the present invention takes into account the drawbacks of the prior art and provides an invention with the main object of providing a method to perform reverse transcription loop-mediated isothermal amplification (RT-LAMP), and LAMP at an isothermal temperature between 25-42° C. which is room temperature to ambient temperatures, more specifically between 25-37° C., and providing a reagent kit thereof.

Further, the method is rapid and enables detection of sample DNA/RNA by LAMP/RT-LAMP within 30-40 mins.

Another object of the present invention is to provide an in vitro method to detect SARS-CoV2 infection using RT-LAMP at an isothermal temperature at room temperature of 25-42° C., more specifically between 25-37° C.

Yet another object of the invention is to provide a reagent kit including primers to perform RT-LAMP at an isothermal temperature at temperature of 25-42° C., more specifically between 25-37° C., to detect SARS-CoV2 infection.

Yet another object of the present invention is to provide a method to perform RT-LAMP and LAMP at temperature between 25-42° C., more specifically between 25-37° C., and eliminating the heating steps to make the technique less expensive and remove the requirement of expensive equipment.

### SUMMARY OF THE INVENTION

In the main embodiment of the invention, the present invention provides a rapid method for amplifying nucleic acid by reverse transcription loop-mediated isothermal amplification (RT-LAMP) at an isothermal temperature between 25-42° C., more specifically at temperatures between 25-37° C. in a single tube assay, the method comprising the steps of:
a) isolating target RNA or DNA from a sample;
b) contacting the target RNA or DNA of step a with a novel RT-LAMP reaction mix tube to generate amplification product, the reaction mix comprising:
   i. AMV Reverse Transcriptase (AMV-RT), RNAse inhibitor, and Random hexamers for converting target RNA to cDNA,
   ii. at least one DNA polymerase having a strand displacement activity for amplifying target DNA or cDNA generated from target RNA,
   iii. nucleic acid strand displacement and chaperone agents,
   iv. nicking enzyme or restriction endonucleases,
   v. 4-8 primers specific against the target with a 3′ end and a 5′ end including forward outward primer, backward outward primer, forward inner primer, and backward inner primer, and
   vi. a buffer solution including deoxyribonucleoside triphosphate (dNTP) mixture; and
c) detecting a signal indicative of amplification of the region of target DNA or RNA.

The novel reaction mix to amplify nucleic acid by RT-LAMP at an isothermal temperature between 25-42° C., more specifically at temperature between 25-37° C. in a single tube assay further comprises DNA polymerases Klenow exo (−/−) and High-fidelity Taq polymerase in a ratio of 5:2 for amplifying target nucleic acid.

The novel reaction mix to amplify nucleic acid by RT-LAMP at an isothermal temperature between 25-42° C., more specifically at temperature between 25-37° C. in a single tube assay further comprises RPA32 (Replication protein A 32) and StpA protein as nucleic acid strand displacement and chaperone agents.

The novel reaction mix to amplify nucleic acid by RT-LAMP at an isothermal temperature between 25-42° C., more specifically at temperature between 25-37° C. in a single tube assay further comprises the restriction enzyme ApaI or BstXI; The novel reaction mix to amplify nucleic acid by RT-LAMP at an isothermal temperature between 25-42° C., more specifically at temperature between 25-37° C. in a single tube assay further comprises Buffer solution containing 20 mM Tris-HCl, 10 mM MgSO₄, 50 mM KCl, 0.1% PEG, 4 mM DTT, 1% DMSO, 1 mM dNTPs each; and a fluorescent dye such as Propidium iodide.

Conventionally RT-LAMP is performed at higher temperatures such as 62-65° C. using Bst DNA polymerase, and AMV Reverse transcriptase. The conventional method also requires higher temperatures for nucleic acid strand displacement, and final denaturation.

The present invention provides a method which utilizes a novel reaction mix which enables RT-LAMP or LAMP amplification of nucleic acids at an isothermal temperature between 25-42° C., more specifically at temperature between 25-37° C. in a single tube assay eliminating the requirement of higher temperatures for nucleic acid strand displacement, amplification, and final denaturation.

DNA polymerases Klenow exo (−/−) and High-fidelity Taq polymerase in the ratio of 5:2 along with nucleic acid strand displacement and chaperone agents, RPA32 (Replication protein A 32) and StpA protein, enable amplification of nucleic acids by RT-LAMP or LAMP at an isothermal temperature between 25-42° C., more specifically at temperature between 25-37° C. in a single tube assay eliminating the requirement of higher temperatures for nucleic acid strand displacement, amplification, and final denaturation. The temperatures between 25-42° C. fall in the range of room temperature to ambient temperature and do not require expensive heating techniques. Rpa 32 is known for its role in DNA replication and StpA is known for its role as RNA chaperone. The binding of Rpa 32 and StpA to the sample DNA/RNA and subsequently produced amplicons after the initiation of the RT-LAMP or LAMP reaction, eliminates the requirement of denaturation of secondary structures at 60-65° C. Other RNA chaperones can be used replacing StpA such as Li, Si, S12.

Further, conventionally, RT-LAMP or LAMP requires around 6-8 primers namely-forward outward primer, backward outward primer, forward inner primer, and backward inner primer, and 2-4 loop primers.

However, the present invention provides a method utilizing a minimal of 4 primers per target instead of 6-8 primers per target unlike the conventional RT-LAMP or LAMP using Bst polymerase. Therefore, multiplex RT-LAMP/LAMP can be performed with more than one target with lesser number of primers required conventionally. One may perform RT-LAMP or LAMP with only the forward outward primer, backward outward primer, forward inner primer, and backward inner primer for a target nucleic acid sequence.

Conventionally, RT-LAMP or LAMP requires forward outward primer, and backward outward primer at a concentration of 0.1-0.5 μM each, and forward inner primer, and backward inner primer at a concentration of 400 pmol to 1.0 M each.

Further, the method requires lower concentrations of primer than existing RT-LAMP or LAMP reactions. Conventionally, RT-LAMP or LAMP reaction mixes contain 0.1-0.5 M each FP/BP, and 400 pmoles each FIP/BIP. However, the present invention provides reaction conditions and reaction mix having 3-10 pmols concentration of FP/BP each, and 12-20 pmol of FIP/BIP each. Hence, it reduces non-spurious amplification and false positives. This is because in case of conventional LAMP more primers are required for strand displacement activity, however, in the present invention it is compensated by the usage of proteins Rpa32 and Stpa.

Further said method using said novel reaction mix enables to perform RT-LAMP assay with 100% sensitivity, and more than 97% specificity.

Additionally, where conventional RT-LAMP and LAMP reactions take more than 1-2 hours to produce amplified product for detection, the present invention provides a method to perform RT-LAMP or LAMP at ambient temperature between 25-37° C., and the time required to complete reaction is as low as 30-45 minutes. At 37° C., the RT-LAMP or LAMP reaction is completed in 30 minutes.

This method is useful to detect target RNA in sample, wherein, the target RNA is genome of infectious microbe in a single tube assay at single isothermal temperature between 25-42° C., more specifically between 25-37° C. Further, the method is useful to detect presence of SARS-CoV2 in a sample in a single tube assay at 37° C. Said method does not require heating at higher temperatures such initial denaturation at 60-65° C. and final inactivation at 80° C., thus making the method extremely useful, and cost-effective.

In another embodiment, the present invention provides a method to perform or LAMP in a single tube assay at an isothermal temperature of room temperature between 25-42° C., more specifically between 25-37° C., to detect target RNA in a sample, the method comprising the steps of:
1. isolating target DNA from a sample;
2. contacting the target DNA a with reaction mix tube to generate amplification product comprising:
   a. Klenow exo (−/−),
   b. High-fidelity Taq polymerase
   c. RPA32 (Replication protein A 32),
   d. StpA protein,
   e. ApaI restriction enzyme,
   f. Buffer mix containing 20 mM Tris-HCl, 10 mM MgSO₄, 4 mM DTT, 1% DMSO, 50 mM KCl, 1 mM dNTPs each, and 4-8 primers specific against the target, and
   g. A fluorescent dye, more specifically, Propidium iodide (PI) dye; or colorimetric DNA binding dye,
3. Adding sample to reaction mix tube and incubating at 25-42° C., more specifically between 25-37° C. for 30-45 minutes; and
4. Observing the reaction mix tube under UV light for fluorescence in case a fluorescent dye is used or observe directly using colorimetric DNA binding dye, where presence of fluorescence or color means detection of target RNA in sample, and absence of fluorescence or color means absence of target RNA in sample.

In another embodiment, the invention provides a method to perform RT-LAMP, with Klenow exo (−/−) supplemented with AMV-RT which is used to convert target RNA to cDNA.

When the reaction is carried out at ambient temperature of 37° C., the time required to complete reaction is as low as 30 mins.

In another embodiment, the invention provides a reaction mix for rapid amplification of nucleic acid by RT-LAMP or LAMP at an isothermal temperature in a single tube assay comprising of:

AMV Reverse Transcriptase (AMV-RT), RNAse inhibitor, and Random hexamers for converting target RNA to cDNA,

at least one DNA polymerase having a strand displacement activity for amplifying target DNA or cDNA generated from target RNA,

nucleic acid strand displacement and chaperone agents,

nicking enzyme or restriction endonucleases,

4-8 primers specific against the target with a 3′ end and a 5′ end including forward outward primer, backward outward primer, forward inner primer, and backward inner primer, and

a buffer solution including deoxyribonucleoside triphosphate (dNTP) mixture;

wherein,

the DNA polymerases in the reaction mix are Klenow exo (−/−) and High-fidelity Taq polymerase at a ratio of 5:2 for amplifying target nucleic acid;

nucleic acid strand displacement and chaperone agents comprising of RPA32 (Replication protein A 32) and StpA protein;

the restriction enzyme is ApaI or BstXI; and the RT-LAMP reaction is performed at an isothermal temperature between 25-42° C., more specifically at temperature between 25-37° C. in a single tube assay eliminating the requirement of higher temperatures for nucleic acid strand displacement, amplification, and final denaturation.

The present invention provides a reaction mix to perform RT-LAMP or LAMP LAMP at an isothermal temperature between 25-42° C., more specifically at temperature between 25-37° C. in a single tube assay, wherein, the primers specific against the target comprise of forward outward primer and backward outward primer at a concentration of 3-10 pmol each, and forward inner primer and backward inner primer at a concentration of 12-20 pmol each.

The present invention further provides buffer solution comprising 20 mM Tris-HCl, 10 mM MgSO₄, 50 mM KCl, 0.1% PEG, 4 mM DTT, 1% DMSO, and 1 mM dNTPs each

In another embodiment the invention provides a reagent kit to perform a single tube LAMP assay at an isothermal temperature of room temperature between 25-42° C., more specifically between 25-37° C., to detect target RNA/DNA in a sample. The reagent kit is a single tube reaction mix comprising of:
a. Klenow exo (−/−),
b. ApaI restriction enzyme,
c. High-fidelity Taq polymerase,
d. RPA32 (Replication protein A 32),
e. StpA protein,
f. Buffer mix containing 20 mM Tris-HCl, 10 mM MgSO₄, 4 mM DTT, 1% DMSO, 50 mM KCl,1 mM dNTPs each, and 4 primers specific against the target, and
g. A fluorescent dye, more specifically, Propidium iodide (PI) dye, or colorimetric DNA binding dye

In order to perform RT-LAMP, Klenow exo (−/−), which also performs RNA polymerase activity, is supplemented with AMV-RT is used to convert target RNA to cDNA.

In yet another embodiment the invention provides a single tube RT-LAMP reaction method at an isothermal temperature between 25-42° C., more specifically at 37° C., to detect SARS-CoV2 in a sample in vitro comprising the steps of:
1. Taking a reaction mix tube containing
   a. Klenow exo (−/−) supplemented with AMV-RT,
   b. ApaI restriction enzyme,
   c. High-fidelity Taq polymerase,
   d. RPA32 (Replication protein A 32),
   e. StpA protein,
   f. Buffer mix containing 20 mM Tris-HCl, 10 mM MgSO₄, 4 mM DTT, 1% DMSO, 50 mM KCl, 1 mM dNTPs each, and 4 primers specific against SARS-CoV2 genome, and g. A fluorescent dye, more specifically, Propidium iodide (PI) dye, or a
   colorimetric DNA binding dye;
2. Adding sample to reaction mix tube and incubating at 37° C. for 30 minutes; and
3. Observing the reaction mix tube under UV light for fluorescence in case a fluorescent dye is used or observe directly using colorimetric DNA binding dye, where presence of fluorescence or color means detection of target DNA/RNA in sample, and absence of fluorescence or color means absence of target DNA/RNA in sample

### Wherein,

the primers comprise:
a. Forward outward primer selected from the group consisting of Seq. ID 1, Seq. ID 5, or Seq. ID 9, or a combination of more than one primer for multiplex RT-LAMP;
b. Backward outward primer selected from the group consisting of Seq. ID 2, Seq. ID 6, or Seq. ID 10, or a combination of more than one primer for multiplex RT-LAMP;
c. Forward inner primer selected from the group consisting of Seq. ID 3, Seq. ID 7, or Seq. ID 11, or a combination of more than one primer for multiplex RT-LAMP; and
d. Backward inner primer selected from the group consisting of Seq. ID 4, Seq. ID 8, or Seq. ID 12, or a combination of more than one primer for multiplex RT-LAMP.
In another embodiment, the invention provides a reaction mix to detect SARS-CoV2 by RT-LAMP at 37° C. within 30 mins, the reaction mix comprising:
   a. Klenow exo (−/−) supplemented with AMV-RT,
   b. ApaI restriction enzyme,
   c. High-fidelity Taq polymerase,
   d. RPA32 (Replication protein A 32),
   e. StpA protein,
   f. Buffer mix containing 20 mM Tris-HCl, 10 mM MgSO₄, 4 mM DTT, 1% DMSO, 50 mM KCl, 1 mM dNTPs each, and 4 primers specific against SARS-CoV2 genome, and
   g. A fluorescent dye, more specifically, Propidium iodide (PI) dye, or colorimetric DNA binding dye;
4. Presence of fluorescence or color means detection of target DNA/RNA in sample and absence of fluorescence means absence of target DNA/RNA in sample

### Wherein,

the primers comprise:
a. Forward outward primer selected from the group consisting of Seq. ID 1, Seq. ID 5, or Seq. ID 9, or a combination of more than one primer for multiplex RT-LAMP;
b. Backward outward primer selected from the group consisting of Seq. ID 2, Seq. ID 6, or Seq. ID 10, or a combination of more than one primer for multiplex RT-LAMP;
c. Forward inner primer selected from the group consisting of Seq. ID 3, Seq. ID 7, or Seq. ID 11, or a combination of more than one primer for multiplex RT-LAMP; and
d. Backward inner primer selected from the group consisting of Seq. ID 4, Seq. ID 8, or Seq. ID 12, or a combination of more than one primer for multiplex RT-LAMP.

### BRIEF DESCRIPTION OF THE DRAWINGS

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 illustrates the Multiple sequence alignment (MSA) of N and S regions of the genomes between the identified HCoV, Bat SARS-CoV, MERS-CoV with their percent identities with respect to SARS-CoV2 genome;

FIG. 2 depicts a schematic of stepwise method for performing RT-LAMP at 37° C., to detect SARS-CoV2 in a patient sample;

FIG. 3A depicts gel image of RT-LAMP reactions at 37° C. using various concentrations of plasmid for E gene;

FIG. 3B depicts gel image of RT-LAMP reactions at 37° C. using various concentrations of plasmid for N gene;

FIG. 4A depicts gel image of RT-LAMP reactions at 37° C. of positive control (PC), RNA sample of RT-PCR+ve Covid patient, cDNA sample of RT-PCR+ve Covid patient, Negative control (NTC), and nucleic acid size ladder (L);

FIG. 4B upper panel depicts the Fluorescence of PI in the reaction mix;

FIG. 4B lower panel depicts the grey scale image of Fluorescence of PI in the reaction mix

FIG. 5 depicts gel image of RT-LAMP reactions at 37° C. of following samples: Buffer composition with Klenow exo(−/−) performed for 30 mins (Lanes 1-2), Lane 1: SARS-CoV2 RT-PCR+ve sample and Lane 2: NTC (no template control);

Buffer composition with Klenow large fragment replacing Klenow exo (−/−) (Lanes 3-8), Lane 3: SARS-CoV2 RT-PCR+ve sample with 30 mins reaction time; Lane 4: SARS-CoV2 RT-PCR+ve sample with 45 mins reaction time; Lane 5: SARS-CoV2 RT-PCR +ve sample with 1 hr reaction time; Lane 6: SARS-CoV2 RT-PCR+ve sample with 2 hrs reaction time; Lane 7: SARS-CoV2 RT-PCR+ve sample with 3 hrs reaction time; Lane 8: NTC with 3 hrs reaction time; and Lane 9: Nucleic acid size ladder; and

FIG. 6 depicts gel image of RT-LAMP reactions at 37° C. of following samples: Buffer composition with Klenow exo(−/−) and ApaI restriction enzyme performed for 30 mins (Lanes 1-2), Lane 1 SARS-CoV2 RT-PCR+ve sample; Lane 2: NTC sample;

Buffer composition with Klenow exo(−/−) and EcoR1 restriction enzyme (Lanes 3-4), Lane 3 SARS-CoV2 RT-PCR+ve sample; Lane 4: NTC sample;

Buffer composition with Klenow exo(−/−) and HindIII restriction enzyme (Lanes 5-6), Lane 5 SARS-CoV2 RT-PCR+ve sample; Lane 6: NTC sample;

Buffer composition with Klenow exo(−/−) and BstXI restriction enzyme (Lanes 7-8), Lane 7 SARS-CoV2 RT-PCR+ve sample; Lane 8: NTC sample;

Lane 9: empty, and Lane 10—Nucleic acid size ladder.

FIG. 7 depicts gel image of RT-LAMP reactions at 37° C. of following samples Lane 1—NTC sample with no template and 5 units Klenow exo (−/−), lane 2—Reaction mix with 5 units of Taq polymerase, lane 3—Nucleic acid size ladder, lane 4-8 units Bst 2.0 polymerase, lane 5-8 units Klenow exo (−/−), lane 6-8 units Klenow fragment, lane 7-8 units Pfu DNA polymerase, lane 8-8 units Q5 polymerase, lane 9-8 Units of Vent polymerase, lane 10-8 units of Vent exo (−/−) polymerase, and lane 11—NTC sample with no template and 8 units of Vent exo (−/−).

FIG. 8 depicts gel image of RT-LAMP reactions at 37° C. of following samples

Lane 1—nucleic acid ladder, lane 2—NTC sample without template, lane 3—reaction mix without Rpa32, lane 4—reaction mix with 25 ng rpa32, lane 5 reaction mix with 50 ng rpa32, lane 6—reaction mix with 100 ng rpa32, lane 8—nucleic acid ladder, lane 9—NTC sample without template, lane 10—reaction mix with 100 ng gp32 (instead of Rpa32), lane 11—reaction mix with 300ng gp32 (instead of Rpa32), lane 13—nucleic acid ladder

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention relates to a rapid method to perform reverse transcription loop-mediated isothermal amplification (RT-LAMP) or LAMP in a single tube assay within 30-45 mins at an isothermal temperature of room temperature 25-42° C., more specifically at 37° C., without the heat inactivation step to detect target RNA/DNA in a sample. Further the invention provides a method to detect SARS-CoV2 genome in a sample by RT-LAMP in a single tube assay within 30-40 mins at an isothermal temperature of room temperature 25-42° C., more specifically at 37° C. without the heat inactivation step. Further the invention provides reaction mix to perform RT-LAMP or LAMP in a single tube assay at an isothermal temperature of room temperature 25-42° C., more specifically at 37° C. without the heat inactivation step.

### Example 1

### Primers to Detect Sars-Cov2 Genome by Rt-Lamp in a Sample

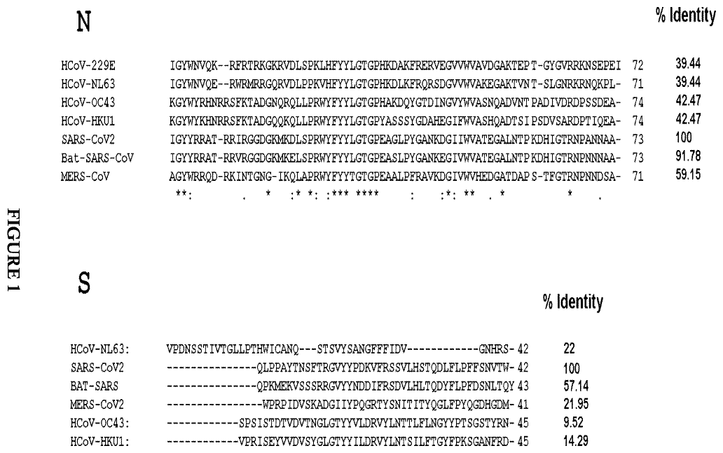

In order to develop the single-tube RT-LAMP assay for detection of all strains of SARS-Cov2 (human SARS-CoV—HCoV), a set of 4 primers were designed against the Nucleocapsid (N), Envelope (E) and Spike (S) region conserved across the known strains of SARS-CoV2 until April 2020, namely—HCoV-229E, HCoV-NL63, HCoV-OC43, and HCoV-HKU1. To make sure there is no cross-reactivity against Bat SARS-CoV, MERS-CoV and other human coronaviruses (HCoVs), only those regions were chosen to design the primers that showed low sequence conservation across these species and strains. FIG. 1 depicts the multiple sequence alignment (MSA) of N and S regions of the genomes between the identified HCoV, Bat SARS-CoV, MERS-CoV with their percent identities with respect to SARS-CoV2 genome.

Table 1 provides the primers designed for RT-LAMP for the N, S, and E regions of SARS-CoV2 genome in such a way that all the known 4 strains of SARS-CoV2 can be detected using these primers. Set of 4 primers were designed against ech region—N, S, and E namely—
F3 (Forward outward primer),
B3 (Backward outer primer),
FIP (Forward inner primer), and
BIP (backward inner primer).

The FIP and BIP also work as the loop primers.

TABLE 1
LAMP primers designed for SARS-COV2 detection

| Seq. | Name | |
|---|---|---|
| ID no. | of Seq. | Sequence |
| 1 | N_F3 | TGGCTACTACCGAAGAGCT |
| 2 | N_B3 | TGCAGCATTGTTAGCAGGAT |
| 3 | N_FIP | TCTGGCCCAGTTCCTAGGTAGTCCAGACGAA |
| | | TTCGTGGTGG |
| 4 | N_BIP | AGACGGCATCATATGGGTTGCACGGGTGCCA |
| | | ATGTGATCT |

E Gene Primers

| | | |
|---|---|---|
| 5 | E_F3 | TTTCGGAAGAGACAGGTAC |
| 6 | E_B3 | AGGAACTCTAGAAGAATTCAGA |
| 7 | E_FIP | CGCAGTAAGGATGGCTAGTGTACGTACTT |
| | | CTTTTTCTTGCTT |
| 8 | E_BIP | TCGATTGTGTGCGTACTGCTGTTTTTAACA |
| | | CGAGAGTAAACGT |

S Gene Primers

| | | |
|---|---|---|
| 9 | S_F3 | ATTACCCCCTGCATAC |
| 10 | S_B3 | AACCAAGTAACATTGGAA |
| 11 | S_FIP | CTGAAAACTTTGTCAGGGTAATACTAATTCTTTCA |
| | | CACGT |
| 12 | S_BIP | ATCCTCAGTTTTACATTCAACTAGAAAGGTAAGAA |
| | | CAAGTC |

To check specific amplification occurred, N, E and S region containing plasmids were used as template for normal PCR with F3 and B3 primers using Kapa Hifi mastermix. S showed expected product size of 100 bp, and no amplification was seen in no template control or negative control (NTC), and human cDNA template (Hu). When both S plasmid and human cDNA were used in the same reaction, amount of amplification remained same, indicating that primers specifically amplified S region without hindrance from human cDNA (figure not shown).

Similarly, PCR with E and N specific primers with E and N plasmids as template yielded specific product at 200 bp, as expected and no amplification was observed in NTC as well as human cDNA template (figure not shown).

### Example 2

### A. RT-LAMP Method To Detect SARS-CoV2 At Room Temperature Between 25-42° C., More Specifically At 37° C.

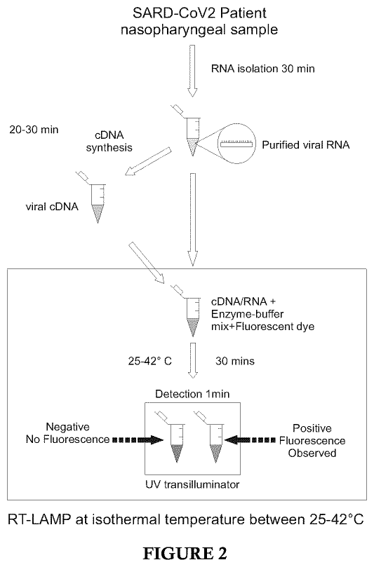

FIG. 2 illustrates a schematic showing stepwise in vitro method to carry out RT-LAMP at room temperature between 25-42° C., more specifically at 37° C., to detect SARS-CoV2 in a patient sample comprising the steps of:
1. Colleting patient sample and isolation of SARS-CoV2 genome (RNA) using standard methods known in prior art;
2. Synthesizing cDNA from the viral RNA using standard protocols known in prior art to form the cDNA/RNA pool;
3. Mixing the cDNA/RNA pool in the single tube RT-LAMP reaction mix comprising of Enzyme, buffer, and florescent dye to carry the RT-LAMP assay;
4. Carrying the RT-LAMP assay in the tube at 25-42° C., more specifically 37° C. for around 30 mins; and
5. Observing the tube under UV trans illuminator to check for fluorescence, wherein, presence of fluorescence means presence of SARS-CoV2 in the patient sample (+ve result), and absence of any fluorescence means absence of SARS-CoV2 in the patient sample (−ve result).

This method requires a unique combination of enzymes and buffers to achieve RT-LAMP at room temperature 25-42° C., more specifically 37° C., eliminating the need of any PCR equipment, or any other expensive equipment other than a water bath to maintain constant temperature. Further, this method also eliminates the requirement of heat inactivation, thus reducing the overall cost of the assay.

Similarly, LAMP at room temperature 25-42° C., more specifically 37° C., to detect DNA in a sample. The same method can be used without the requirement of generation of cDNA.

### B. RT-LAMP Reagent kit or Reaction Mix Composition To Detect SARS-CoV2 At Room Temperature Between 25-42° C., More Specifically At 37° C.

To conduct RT-LAMP at room temperature 25-42° C., more specifically 37° C., a unique reaction mix is required comprising of:
a. Klenow exo (−/−) or Klenow large fragment,
b. ApaI restriction enzyme,
c. High-fidelity Taq polymerase,
d. RPA32 (Replication protein A 32),
e. StpA protein,
f. 4 primers specific against the target region of SARS-CoV2 genome
g. Buffer mix containing 20 mM Tris-HCl, 10 mM MgSO₄, 4 mM DTT, 1% DMSO, 50 mM KCl, 1 mM dNTPs each, and
h. A fluorescent dye, more specifically, Propidium iodide (PI) dye. cDNA is prepared from RNA in the sample using Klenow exo−/− supplemented with AMV-RT. This cDNA is added to the above reaction mix to carry out RT-LAMP.

Table 2 provides a comparison of reaction mix components of present invention and that of conventional RT-LAMP/LAMP reaction mix.

TABLE 2

| | | | |
|---|---|---|---|
| Present | | | |
| invention | Amount/ | Conventional LAMP | Amount/ |
| Reaction Mix | Concentration | reaction mix | Concentration |

Enzymes/Proteins

| | | | | |
|---|---|---|---|---|
| LAMP enzyme | Klenow exo−/− | 5 U | Bst 2.0 WarmStart ® | 8 U |
| | | | DNA Polymerase | |
| Restriction | ApaI | 0.2 U | — | — |
| Enzyme | | | | |
| Other | High fidelity | 2 U | — | — |
| polymerase | Taq Pol | | | |
| Nucleic acid | Rpa32 | 100 ng | — | — |
| binding proteins | StpA | 50 ng | | |

Buffer composition

| | | | | |
|---|---|---|---|---|
| | Tris-HCl | 20 mM | Tris-HCl | 20 mM |
| | MgSO4 | 10 mM | MgSO4 | 2 mM |
| | KCl | 50 mM | KCl | 50 mM |
| | DTT | 4 mM | DTT | 1 mM |
| | PEG | 0.1% | | — |
| | DMSO | 1% | | — |
| | | — | Tween 20% | 0.1% |
| | | — | DMSO | 0.1 mM |
| | | | (NH4)2SO4 | 10 mM |
| Primers | 4 nos (F3, B3, | 3-10 pmol | 6 to 8 nos (F1-3, | 0.1-0.5 μM |
| | FIP, and BIP) | F3/R3; | B1-3, FIP, BIP) | each F1-3/R1-3 |
| | | 12-20 pmol | | 400 pmoles |
| | | FIP/BIP each | | each FIP/BIP |
| | dNTPs | 1 mM each | dNTPs | 1.5 mM each |
| Fluorescent Dye | PI | ~negligble | May or may | |
| | | | not be | |

Additional for RT-LAMP

| | | | |
|---|---|---|---|
| RNAse inhibitor | 2U | RNAse inhibitor | 2U |
| Random hexamers | 10 pmol | Random hexamers | |
| AMV RT | 5U | AMVRT | 8U |

RT-LAMP at room temperature 35-42° C., more specifically 37° C., was carried out using Klenow-exo (−/−), a strand displacement enzyme having polymerase activity and also reported to have reverese transcriptase activity. Klenow exo (−/−) was supplemented with a reverse transcriptase (not used if starting with cDNA), restriction enzyme and the above-mentioned buffer components, allowing reverse transcription, amplification and detection in one-tube. The primer concentrations and buffer components were optimized to eliminate non-specific amplification, a problem generally observed in other LAMP procedures.

To first test the RT-LAMP/LAMP reaction mix, LAMP was performed with plasmid constructs for N, and E genes (DNA sample) at 37° C. NTC is no template control or negative control for that set of reactions with highest conc of all reaction mix components being tested to capture any spurious amplification.

RT-LAMP/LAMP reaction mix tubes containing novel single-tube reaction mix were taken. Each tube had 25ul of reaction mix as provided in Table 2, and 0.5 ul each of N gene set and E gene set of primers (total 8 primers-Seq. ID Nos. 1-8), and PI dye from working stock. This reaction mix comprises LAMP primer set against 2 different regions, N gene and E gene, of the SARS-CoV2 genome in a single reaction tube, thus making it a multiplex RT-LAMP. Multiplexing generally increases the chances of detection of target by RT-LAMP assay.

Plasmid concentrations ranging from 0.05-50 ng was tested for E gene, and concentrations ranging from 0.005-50 ng was tested for N gene.

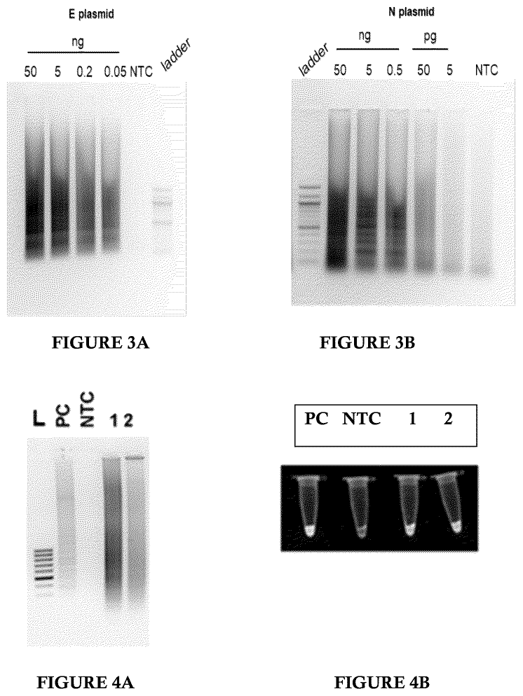

As depicted in FIG. 3A and FIG. 3B, reaction sample which did not have any template or target RNA (no template control-NTC) did not show any amplification, whereas, all the plasmid samples showed amplification. Further, increasing concentrations of plasmid increased the amplification suggesting specificity of the primers to the gene in the plasmids.

Further, RT-LAMP was carried out using the RT-LAMP reaction mix at 37° C. using S, E, and N primer sets with cDNA samples obtained from cultured virus (virus+Con), SARS-COV2 positive patient sample and human tissue. Additionally, to check interference from human cDNA, the cDNA from patient sample was complexed with human and RT-LAMP was carried out at 37° C. Specific LAMP products starting from 200 bp were observed for positive viral control, SARS-CoV2 positive patient samples, as well as patient cDNA complexed with human cDNA. NTC and human cDNA did not show any amplification. Thus, the primer specificity was established.

Post amplification, detection in same tube was performed using DNA-binding dyes Propidium Iodide (PI), Acridine Orange (AO) and Ethidium bromide (EtBr), dyes that fluorescence under UV excitation. In all three, amplification was observed as enhanced fluorescence compared to that in NTC. PI showing bright pink fluorescence was consistent in recapitulating the results as confirmed by agarose gel electrophoresis. EtBr also worked efficiently as PI (result not shown).

As results were very satisfactory with PI at room temperature, it was chosen as the dye for the tests.

### C. RT-LAMP to Detect SARS-CoV2 in Human Sample at Room Temperature Using RT-LAMP Reaction Mix Between 25-42° C., More Specifically At 37° C.

Multiplex RT-LAMP reaction mix tubes containing 25ul of reaction mix containing components as provided in Table 2, and 0.5 ul each of N gene set and E gene set of primers (total 8 primers-Seq. ID Nos. 1-8), Apa I restriction enzyme, Taq polymerase, and PI dye. This reaction mix comprises LAMP primer set against 2 different regions, N gene and E gene, of the SARS-CoV2 genome in a single reaction tube, thus making it a multiplex RT-LAMP. NTC is no template control or negative control for that set of reactions with highest conc of all reaction mix components being tested to capture any spurious amplification. 5 ul each of RNA isolated from one CoVID-19 patient (RT-PCR+ve) was added to reaction tube 1 and 5 ul of cDNA from RNA of same patient was added to reaction tube 2. 5ul of positive control RNA (isolated from cultured SARS-CoV2 virus) was added to tube labelled PC. 5ul of sterile water was added to tube labelled NTC (negative control). The tubes were kept floating in a heating water bath at 37° C. for 30 mins. Then the tubes were taken out and kept under UV trans illuminator and imaged. As depicted in FIG. 4B the PC, 1, and 2 labelled tubes showed high pink fluorescence, indicating positive result, while NTC showed very low background fluorescence, indicating negative result. To confirm this result, gel electrophoresis was performed with the reaction mixes. As depicted in FIG. 4A, the reaction mixes from PC, 1, and 2 tubes showed amplification and NTC did not show any amplification which mirrored the result of colorimetric test. Hence, the test was confirmed to be positive for samples 1 & 2.

Patient samples, previously reported to be positive by RT-PCR with commercial primers, were used for checking the validity of the LAMP test. RT-LAMP at 37° C. was tested for one-tube reaction starting from RNA isolated from SARS-CoV2 patient samples with N and E multiplexed primers. It showed that RNaseP primers also worked at 37° C. using the RT-LAMP reaction mix and can be used as a control for testing sample quality. This data cross compared with RT-PCR test results for even patient samples showing high Ct value above 35 in real-time PCR. Totally 80+samples had been tested.

Sensitivity and specificity of test has been found to be 100% and 97% respectively.

### Example 3

Efficiency of Klenow exo (−/−) in the reaction mix to perform RT-LAMP at 37° C. over Klenow large fragment

The Klenow fragment is a large protein fragment produced when DNA polymerase I from E. coli is enzymatically cleaved by the protease subtilisin, called as Klenow large fragment. It retains the 5′→3′ polymerase activity and the 3′→5′ exonuclease activity, but does not have the 5′→3′ exonuclease activity. Whereas, the Klenow exo (−/−) fragment is a shorter fragment that retains 5′→3′ polymerase activity, but lack any exonuclease activity (5′→3′ or 3′→5′).

Both these enzymes were tested for their efficiency to perform RT-LAMP at 37° C. A sample of SARS-CoV2 positive patient was taken and added to 25 μl RT-LAMP reaction mix containing enzymes either Klenow exo (−/−) fragment or Klenow large fragment as described in Table 2, and buffer composition of 20 mM Tris-HCl, 10 mM MgSO₄, 4 mM DTT, 1% DMSO, 50 mM KCl, 1 mM dNTPs each, and 0.5 ul each of N gene set and E gene set of primers (total 8 primers-Seq. ID Nos. 1-8), Apa I restriction enzyme, and PI dye. NTC is no template control or negative control for that set of reactions with highest conc of all reaction mix components being tested to capture any spurious amplification.

Tube with SARS-CoV2 sample with Klenow exo (−/−) fragment was incubated for 30 mins at 37° C. NTC sample with Klenow exo (−/−) fragment and reaction mix was also incubated for 30 mins at 37° C. Whereas, five reaction tubes containing Klenow large fragment and reaction mix with SARS-CoV2 sample were incubated at 37° C. for 30 mins, 45 mins, 1 hour, 2 hours, and 3 hours respectively. NTC sample with Klenow large fragment and reaction mix was incubated at 37° C. for 3 hours. All the samples were run on a gel along with DNA ladder.

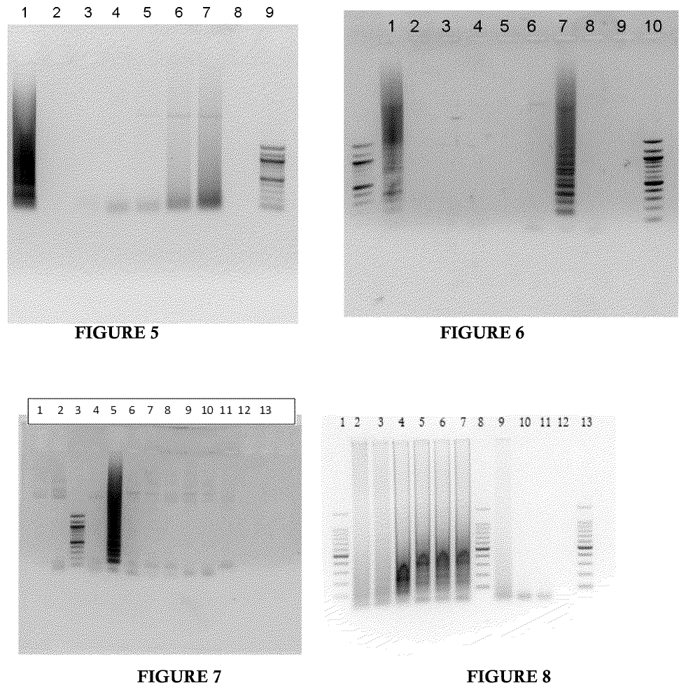

As depicted in FIG. 5, Klenow exo (−/−) fragment showed amplification in SARS-CoV2 sample (lane 1), and did not show any amplification in NTC sample (lane 2), within 30 mins of reaction time. Whereas, Klenow large fragment showed a very weak amplification in 30 mins for SARS-CoV2 sample (lane 3), which gradually increased from reaction times 45 mins to 3 hours (lanes 4-5). Even after 3 hours the amount of amplification visibly was significantly less with Klenow large fragment compared to 30 min reaction with Klenow exo (−/−) fragment.

This showed the efficiency of Klenow exo (−/−) fragment over Klenow large fragment to perform RT-LAMP at 37° C.

### Example 4

### Requirement of Restriction Enzyme Apa I and BstXI in the Reaction Mix to Perform RT-LAMP at 37° C.

Various Type II restriction endonucleases were tested for their efficiency in RT-LAMP at 37° C. ApaI and BstXI are both type II restriction endonucleases that are not sensitive to dam methylation. Further, HindIII and EcoR1 was also tested.

RT-LAMP reaction mixes containing Klenow exo (−/−) as described in Table 2, and buffer composition of 20 mM Tris-HCl, 10 mM MgSO₄, 4 mM DTT, 1% DMSO, 50 mM KCl, 1 mM dNTPs each, and 0.5 ul each of N gene set and E gene set of primers (total 8 primers-Seq. ID Nos. 1-8), and PI dye with either ApaI, BstXI, HindIII, or EcoR1 were prepared. NTC is no template control or negative control for that set of reactions with highest conc of all reaction mix components being tested to capture any spurious amplification.

SARS-CoV2 sample was tested for RT-LAMP using the above reaction mixes containing different restriction enzymes. All samples were incubated at 37° C. for 30 mins. As depicted in FIG. 6, replacement of ApaI with EcoR1 or HindIII did not result in LAMP reaction. While replacement of ApaI with BstXI showed successful LAMP amplification at 37° C.

### Example 5

### Analysis of Requirement of Klenow Exo (−/−) in the Reaction Mix to Perform RT-LAMP at 37° C.

The understand if RT-LAMP or LAMP can be performed at 37° C. using any other known LAMP enzymes the following experimental set up was made:

RT-LAMP reaction mixes containing Klenow exo (−/−) as described in Table 2, and buffer composition of 20 mM Tris-HCl, 10 mM MgSO₄, 4 mM DTT, 1% DMSO, 50 mM KCl, 1 mM dNTPs each, and 0.5 ul each of N gene set and E gene set of primers (total 8 primers-Seq. ID Nos. 1-8), and PI dye with either ApaI, was used with SARS-CoV2 RT-PCR+ve RNA template, and the 5 units of Klenow exo (−/−) enzyme was replaced several conventionally used RT-LAMP enzymes. NTC is no template control or negative control for that set of reactions with highest conc of all reaction mix components being tested to capture any spurious amplification.

As depicted in FIG. 7 gel picture of amplified product after 30 mins reaction at 37° C., only reaction in the presence of 5 units Klenow exo (−/−) (lane 5) showed amplification, whereas replacement of 5 units Klenow exo (−/−) with:

With no enzyme (lane 1), 5 units of Taq polymerase (lane 2), 8 units Bst 2.0 polymerase (lane 4), 8 units Klenow fragment (lane 6), 8 units Pfu DNA polymerase (lane 7), 8 units Q5 polymerase (lane 8), 8 Units of Vent polymerase, 8 units of Vent exo (−/−) polymerase, and NTC-did not show any amplification.

This clearly establishes that only Klenow exo (−/−) enables to perform RT-LAMP or LAMP at 37° C., at ambient temperature.

### Example 6

Analysis of Range of Temperature at which RT-LAMP can be Performed at Ambient Temperature

To analyze the best temperature and the range of temperature at which RT-LAMP can be performed at ambient temperatures and room temperature, RT-LAMP reaction mixes containing Klenow exo (−/−) as described in Table 2, and buffer composition of 20 mM Tris-HCl, 10 mM MgSO₄, 4 mM DTT, 1% DMSO, 50 mM KCl, 1 mM dNTPs each, and 0.5 ul each of N gene set and E gene set of primers (total 8 primers-Seq. ID Nos. 1-8), and PI dye with either ApaI, was used with SARS-CoV2 RT-PCR+ve RNA template were incubated at different temperatures ranging from 25-42° C.

TABLE 3
Comparison of time taken for completing RT-LAMP reaction
at ambient temperatures ranging from 25-42° C.

| Temperature | Time | No. of | |
|---|---|---|---|
| (deg C.) | (min) | samples | LOD |
| 25 | 45 | 25 | 10-100 |
| 30-35 | 30 | 50 | 10-100 |
| 37 | 30 | >100 | 10-100 |
| 42 | 30 | 15 | 10-100 |

The results in Table 3 clearly show that the most rapid method is to perform the RT-LAMP at 37° C. where the reaction is completed in 30 minutes. But the same reaction can easily be performed at room temperature 25° C. which takes 45 minutes, also at other ambient temperatures ranging between 30-42° C. where the reaction is completed in 30 minutes where the limit of detection (LOD) in each case is 10-100.

Hence, RT-LAMP can be performed in a range of ambient temperatures and room temperature between 25-42° C. without any requirement of expensive heating equipment.

### Example 7

### Analysis of Requirement of RPA32 for Performing RT-LAMP at 37° C.

The requirement of Rpa32 for performing RT-LAMP at 37° C. using the reaction mix was tested. RT-LAMP reaction mixes containing Klenow exo (−/−) as described in Table 2, and buffer composition of 20 mM Tris-HCl, 10 mM MgSO₄, 4 mM DTT, 1% DMSO, 50 mM KCl, 1 mM dNTPs each, and 0.5 ul each of N gene set and E gene set of primers (total 8 primers-Seq. ID Nos. 1-8), and PI dye with either ApaI, was used with SARS-CoV2 RT-PCR+ve RNA template. NTC is no template control or negative control for that set of reactions with highest conc of all reaction mix components being tested to capture any spurious amplification.

As depicted in FIG. 8, when the reaction mix contained 25 ng Rpa32 (lane 4), 50 ng Rpa32 (lane 5), or 100 ng Rpa32 was present, target nucleic acid amplification was observed. However, when there was no target nucleic acid (NTC) (lane 2), or Rpa32 was completed absent (Lane 3), or no target nucleic acid (NTC) with 100 ng GP32 (lane 9) instead of Rpa32, or with 100 ng GP32 (instead of rpa32) (lanel0), or with 300ng GP32 (instead of rpa32) there was amplification seen.

This showed that the replacement of Rpa32 with another single-stranded DNA binding protein-GP32 severally affected the reaction.

Thus, these experimental details clearly show that RT-LAMP at 37° C. can be performed with Rpa32 as DNA binding protein.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

We claim:

1. A rapid method for amplifying nucleic acid by reverse transcription loop-mediated isothermal amplification (RT-LAMP) or loop-mediated isothermal amplification (LAMP) at an isothermal temperature in a single tube assay comprising the steps of:
a) Isolating target RNA or DNA from a sample;
b) contacting the target RNA or DNA of step a with a RT-LAMP reaction mix tube to generate amplification product, the reaction mix comprising:
AMV Reverse Transcriptase (AMV-RT), RNAse inhibitor, and Random hexamers for converting target RNA to cDNA,
at least one DNA polymerase having a strand displacement activity for amplifying target DNA or cDNA generated from target RNA,
nucleic acid strand displacement and chaperone agents,
nicking enzyme or restriction endonucleases,
4-8 primers specific against the target with a 3′ end and a 5′ end including forward outward primer, backward outward primer, forward inner primer, and backward inner primer, and
a buffer solution including deoxyribonucleoside triphosphate (dNTP) mixture; and
c) detecting a signal indicative of amplification of the region of target DNA or RNA;
wherein,
the DNA polymerases in the reaction mix are Klenow exo (−/−) and High-fidelity Taq polymerase in a ratio of 5:2 for amplifying target nucleic acid;
nucleic acid strand displacement and chaperone agents comprising of RPA32 (Replication protein A 32) and StpA protein;
the restriction enzyme is ApaI or BstXI;
the RT-LAMP reaction is performed at an isothermal temperature between 25-42° C., more specifically at temperature between 25-37° C. in a single tube assay eliminating the requirement of higher temperatures for nucleic acid strand displacement, amplification, and final denaturation; and
Sensitivity of RT-LAMP is 100%, and specificity is more than 97%.

2. The rapid method for amplifying nucleic acid as claimed in claim 1, wherein, the primers specific against the target comprise of forward outward primer and backward outward primer at a concentration of 3-10 pmol each, and forward inner primer and backward inner primer at a concentration of 12-20 pmol each.

3. The rapid method for amplifying nucleic acid as claimed in claim 1, wherein, the buffer solution comprises 20 mM Tris-HCl, 10 mM MgSO₄, 50 mM KCl, 0.1% PEG, 4 mM DTT, 1% DMSO, and 1 mM dNTPs each.

4. The rapid method for amplifying nucleic acid as claimed in claim 1, wherein, the primers are specific for detecting SARS-CoV2 at an isothermal temperature between 25-37° C. comprising of:
a. Forward outward primer selected from the group consisting of Seq. ID 1, or Seq.
ID 5, or a combination of more than one primer for multiplex RT-LAMP;
b. Backward outward primer selected from the group consisting of Seq. ID 2, or Seq.
ID 6, or a combination of more than one primer for multiplex RT-LAMP;
c. Forward inner primer selected from the group consisting of Seq. ID 3, or Seq. ID 7, or a combination of more than one primer for multiplex RT-LAMP; and
d. Backward inner primer selected from the group consisting of Seq. ID 4, or Seq.
ID 8, or a combination of more than one primer for multiplex RT-LAMP.

5. A reaction mix for rapid amplification of nucleic acid by RT-LAMP or LAMP at an isothermal temperature in a single tube assay comprising of:
AMV Reverse Transcriptase (AMV-RT), RNAse inhibitor, and Random hexamers for converting target RNA to cDNA,
at least one DNA polymerase having a strand displacement activity for amplifying target DNA or cDNA generated from target RNA,
nucleic acid strand displacement and chaperone agents,
nicking enzyme or restriction endonucleases, 4-8 primers specific against the target with a 3′ end and a 5′ end including forward outward primer, backward outward primer, forward inner primer, and backward inner primer, and
a buffer solution including deoxyribonucleoside triphosphate (dNTP) mixture;
wherein,
the DNA polymerases in the reaction mix are Klenow exo (−/−) and High-fidelity Taq polymerase at a ratio of 5:2 for amplifying target nucleic acid;
nucleic acid strand displacement and chaperone agents comprising of RPA32 (Replication protein A 32) and StpA protein;
the restriction enzyme is ApaI or BstXI; and
the RT-LAMP reaction is performed at an isothermal temperature between 25-42° C., more specifically at temperature between 25-37° C. in a single tube assay eliminating the requirement of higher temperatures for nucleic acid strand displacement, amplification, and final denaturation.

6. The reaction mix as claimed in claim 5, wherein, the primers specific against the target comprise of forward outward primer and backward outward primer at a concentration of 3-10 pmol each, and forward inner primer and backward inner primer at a concentration of 12-20 pmol each.

7. The reaction mix as claimed in claim 5, wherein, the buffer solution comprises 20 mM Tris-HCl, 10 mM MgSO₄, 50 mM KCl, 0.1% PEG, 4 mM DTT, 1% DMSO,
and 1 mM dNTPs each

8. The reaction mix as claimed in claim 5, wherein, the primers are specific for detecting SARS-CoV2 at an isothermal temperature between 25-37° C. comprising of:
a) Forward outward primer selected from the group consisting of Seq. ID 1, or Seq.
ID 5, or a combination of more than one primer for multiplex RT-LAMP;
b) Backward outward primer selected from the group consisting of Seq. ID 2, or Seq. ID 6, or a combination of more than one primer for multiplex RT-LAMP;
c) Forward inner primer selected from the group consisting of Seq. ID 3, or Seq. ID 7, or a combination of more than one primer for multiplex RT-LAMP; and
d) Backward inner primer selected from the group consisting of Seq. ID 4, or Seq.
ID 8, or a combination of more than one primer for multiplex RT-LAMP.
